(19) United States
(12) Patent Application Publication
(10) Pub. No.: US 2009/0081259 A1
(43) Pub. Date: Mar. 26, 2009

(54) 1-Aminocyclohexane derivatives for the treatment of multiple sclerosis, emotional lability and pseudobulbar affect

(75) Inventors: Jeffrey Jonas, Hoboken, NJ (US); Allison Mann, Fort Lee, NJ (US); Christopher Graham Raphael Parsons, Nidderau (DE); Wojciech Danysz, Nidderau (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: MERZ PHARMA GMBH & CO. KGAA, FRANKFURT AM MAIN (DE)

(21) Appl. No.: 12/221,574

(22) Filed: Aug. 5, 2008

Related U.S. Application Data
(62) Division of application No. 11/315,581, filed on Dec. 21, 2005, now abandoned.
(60) Provisional application No. 60/638,423, filed on Dec. 22, 2004.

(51) Int. Cl. A61K 31/13

(52) U.S. Cl. 424/278.1; 514/579

(57) ABSTRACT

The present invention relates to the treatment of individuals diagnosed with multiple sclerosis, emotional lability or pseudobulbar affect comprising administering to said individual an effective amount of a 1-aminocyclohexane derivative, namely memantine or neramexane.

### FIELD OF THE INVENTION

The present invention relates to the treatment of individuals diagnosed with multiple sclerosis, emotional lability or pseudobulbar affect comprising administering to said individual an effective amount of a 1-aminocyclohexane derivative.

### BACKGROUND OF THE INVENTION

This invention relates to methods of treating patients suffering from multiple sclerosis (MS) or emotional problems that occur in relation to neurodegenerative diseases or to brain damage such as caused by stroke or head injury.

Multiple sclerosis is a disease of the central nervous system and results in the progressive loss of certain body functions and physical abilities. MS is a progressive and usually fluctuating disease with exacerbations (patients feeling worse) and remissions (patients feeling better) over many decades. Eventually, in most patients, remissions do not reach baseline levels and permanent disability and sometimes death occurs. The cause of MS is unknown.

Emotional lability (EL) is a disease of the central nervous system whereby the patient experiences rapid and sizeable mood changes that can be easily provoked and can rapidly disappear. It is thought to result from an underlying defect in the voluntary control (cognitive) of emotional reactions. Pseudobulbar affect (PBA) is a more severe form of emotional lability in which there are uncontrollable episodes of laughing and/or crying that are unpredictable and seem to have little or no relationship to actual events or the individual's actual feelings.

Various therapeutic approaches have been used in the treatment of MS. Immunomodulators, including glatiramer acetate, have been demonstrated to be effective in improving symptoms associated with MS.

NMDA receptor antagonists potentially have a wide range of therapeutic applications in numerous CNS disorders such as acute neurodegeneration associated with stroke and trauma, chronic neurodegeneration associated with Parkinson's diseases such as Alzheimer's disease, Huntington's disease, amyotrophic lateral sclerosis (ALS), epilepsy, drug dependence, depression, anxiety, and chronic pain (see: Parsons et al., Drug News Perspect., 1998, 11:523-533; Parsons et al., 1999, supra; Jentsch and Roth, Neuropsychopharmacology, 1999, 20: 201-205; Doble, Therapie, 1995, 50: 319-337). Many NMDA receptor antagonists identified to date produce highly undesirable side effects at doses within their putative therapeutic range. Thus, clinical trials failed to support good therapeutic utility due to numerous side effects for such NMDA receptor antagonists as Dizocilpine ((+)MK-801; (+)-5-methyl-10,11-dihydro-5H-dibenzocyclohepten-5,10-imine maleate), Cerestat (CNS-1102), Licostinel (ACEA 1021), Selfotel (CGS-19755), and D-CPP-ene (Leppik, Epilepsia, 1998, 39 (Suppl 5):2-6; Sveinbjornsdottir et al., Epilepsia, 1993, 34:493-521; SCRIP 2229/30, 1997, p. 21).

Memantine (1-amino-3,5-dimethyl adamantane) and neramexane (1-amino-1,3,3,5,5-pentamethylcyclohexane), two NMDA receptor antagonists and analogs of 1-aminocyclo-hexane, prevent the pathological activation of NMDA receptors but allow their physiological activity. Both memantine and neramexane, as well as some other 1-aminoalkyl-cyclohexanes, are systemically-active noncompetitive NMDA receptor antagonists having moderate affinity for the receptor. They exhibit strong voltage dependent characteristics and fast blocking/unblocking kinetics (Parsons et al., 1999, supra; Winblad et al., Int. J. Geriat. Psychiatry, 1999, 14:135-146; Rogawski, Amino Acids, 2000, 19: 133-49; Danysz et al., Curr. Pharm. Des., 2002, 8:835-43; Jirgensons et. al., Eur. J. Med. Chem., 2000, 35: 555-565). These compounds dissociate from the NMDA receptor channels much more rapidly than the high affinity NMDA receptor antagonists such as (+)MK-801 and attenuate disruption of neuronal plasticity produced by tonic overstimulation of NMDA receptors probably by causing an increase of the signal-to-noise ratio. Due to their relatively low affinity for the receptor, strong voltage dependency and fast receptor unblocking kinetics, these compounds are essentially devoid of the side effects of other NMDA receptor antagonists at doses within the therapeutic range.

Memantine, neramexane as well as other 1-aminoalkylcyclohexanes have been suggested to be useful in alleviation of various progressive neurodegenerative disorders such as dementia in AD, Parkinson's disease, and spasticity (see U.S. Pat. Nos. 5,061,703; 5,614,560; and 6,034,134; Parsons et al., 1999, supra; Möbius, ADAD, 1999,13:S172-178; Danysz et al., Neurotox. Res., 2000, 2:85-97; Winblad and Poritis, Int. J. Geriatr. Psychiatry, 1999, 14:135-146; Görtelmeyer et al., 1992, supra; Danysz et al., Curr. Pharm. Des., 2002, 8:835-843; Jirgensons et. al., Eur. J. Med. Chem., 2000, 35: 555-565). These diseases result from disturbances of glutamatergic transmission, i.e., the excessive influx of calcium through NMDA receptor channels, leading to the destruction of brain cells in specific brain areas (Choi, J. Neurobiol., 23: 1261-1276, 1992; Rothman and Olney, Trends Neurosci., 10: 299, 1987; Kemp et al., Trends Pharmacol. Sci., 8: 414, 1987). Treatment of adult rats with memantine has been shown to enhance the formation of hippocampal long-term potentiation, increase the durability of synaptic plasticity, improve spatial memory abilities, and reverse the memory impairment produced by NMDA receptor agonists (Barnes et al., Eur. J. Neurosci., 1996; 8:565-571; Zajaczkowski et al., Neuropharm, 1997, 36:961-971). 1-Aminocyclohexane derivatives, and specifically memantine, have also been suggested to be useful in the treatment of AIDS dementia (U.S. Pat. No. 5,506,231), neuropathic pain (U.S. Pat. No. 5,334,618), cerebral ischemia (U.S. Pat. No. 5,061,703), epilepsy, glaucoma, hepatic encephalopathy, multiple sclerosis, stroke, and tardive dyskinesia (Parsons et al., 1999, supra). Furthermore, relatively high doses of memantine and neramexane were shown to selectively block thermal hyperalgesia and mechanical allodynia in some models of chronic and neuropathic pain without obvious effects on motor reflexes. 1-Aminocyclohexane derivatives were also demonstrated to possess immunomodulatory, antimalarial, anti-Borna virus, and anti-Hepatitis C activities (see, e.g., U.S. Pat. No. 6,034,134 and references cited therein).

U.S. Pat. No. 5,206,248 ('248) describes the treatment of emotional lability by administration of a non-addictive analog of morphine, dextromethorphan or dextrorphan, as a mono-therapy or in combination with quinine. One known mechanism of action for Dextromethorphan includes as a NMDA receptor antagonist. PCT Patent Application 2004/006930 ('930) describes the treatment of neurological disorders including multiple sclerosis, emotional lability and pseudobulbar affect using a combination therapy of a non-addictive analog of morphine, dextromethorphan or dextrorphan, in combination with quinine.

Memantine has been investigated to treat neurological deficits in Lewis rat experimental autoimmune encephalomyelitis (EAE). (Bolton, J. Pharmacol Exp Ther. 2002, July; 302(1): 50-57; Wallstrom, J. Neurol. Sci. 1996, 137, 89-96). Although EAE is the typical animal model of MS, it is not multiple sclerosis, nor is EAE a single disease in a single species. EAEs different forms only resemble the various forms, symptoms and stages of MS. Those forms and symptoms vary greatly depending on the species of rodent used. Additionally, the Lewis rat experiments only studied memantine only to a limited extent. The Bolton study only looked at motor symptoms and histology of the cerebellum, spinal cord, and medulla. Whereas the Wallstrom study merely suggested the therapeutic effect of memantine in the EAE model was not due to dampened CNS inflammation secondary to immune cell activation. The effect of memantine on frontal cortex or supranuclear structures circuitry was not assessed. In addition, these EAE experiments are not suitable for evaluating pseudobulbar affect which occurs in the later stages of MS. The animals were sacrificed after only 2 days. Moreover, an EAE model is generally not applicable for testing emotional lability or pseudobulbar affect symptoms which most notably include uncontrollable emotional expressions.

Memantine has been shown effective for the treatment of acquired pendular nystagmus, a physical symptom of the underlying condition of multiple sclerosis. (Curr. Treat Options Neurol. 1999 March;1(1):68-73) Yet, this invention demonstrates for the first time that the clinical administration of memantine, a 1-aminocyclohexane derivative, is effective for the treatment of cognitive dysfunction associated with multiple sclerosis. It will be shown that MS patients with cognitive impairment treated with memantine demonstrate unexpected improvement in performance on a neuropsychological test battery as compared to placebo treated patients.

### SUMMARY OF THE INVENTION

The present invention relates to the treatment of individuals diagnosed with multiple sclerosis, emotional lability or pseudobulbar affect comprising administering to said individual an 1-aminocyclohexane derivative.

In a specific embodiment of the instant invention, the 1-aminocyclohexane is selected from memantine, neramexane, and derivatives thereof, including pharmaceutically acceptable salts and analogs of these active agents.

A further aspect of the invention relates to the treatment of individuals diagnosed with multiple sclerosis, emotional lability or pseudobulbar affect comprising administering to said individual an 1-aminocyclohexane derivative and an additional pharmaceutical agent which has been shown to be effective in treating MS, EL and PBA.

A further aspect of the invention relates to the treatment of individuals diagnosed with multiple sclerosis, emotional lability or pseudobulbar affect comprising administering to said individual an 1-aminocyclohexane derivative and an immunomodulator.

In a specific embodiment of the instant invention the 1-aminocyclohexane is selected from memantine, neramexane, and derivatives thereof, including pharmaceutically acceptable salts and analogs of these active agents, and the immunomodulator is selected from glatiramer acetate and derivatives thereof, including pharmaceutically acceptable salts and analogs of these active agents.

A further aspect of the invention includes a pharmaceutical composition comprising a therapeutically effective amount of a 1-aminocyclohexane, alone or in combination with other therapies for MS, EL and PBA and, optionally, at least one pharmaceutically acceptable carrier or excipient.

A further aspect of the invention includes a pharmaceutical composition comprising a therapeutically effective amount of a 1-aminocyclohexane, an immunomodulator and at least one pharmaceutically acceptable carrier or excipient.

A further aspect of the invention includes a pharmaceutical composition comprising a therapeutically effective amount of a 1-aminocyclohexane derivative in an immediate or modified release formulation.

A further aspect of the invention includes a pharmaceutical composition comprising a therapeutically effective amount of a 1-aminocyclohexane derivative and an immunomodulator in an immediate or modified release formulation.

### BRIEF DESCRIPTION OF THE DRAWINGS

FIGS. 1A and 1B depict the protocol for EAE inducement and symptomatic progression in animal models.

FIG. 2 is a plot of the resistance to flexion (N) over time post-injection (minutes). The data points reflect the neramexane dose at 3.1 mg/kg.

FIG. 3 is a plot of the resistance to flexion (N) over time post-injection (minutes). The data points reflect the neramexane dose at 6.2 mg/kg.

FIG. 4 is a plot of the resistance to flexion (N) over time post-injection (minutes). The data points reflect the neramexane dose at 12.3 mg/kg.

### DETAILED DESCRIPTION OF THE INVENTION

The term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The term “analog” or “derivative” is used herein in the conventional pharmaceutical sense, to refer to a molecule that structurally resembles a reference molecule (such as 1-aminocyclohexane), but has been modified in a targeted and controlled manner to replace one or more specific substituents of the referent molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate mammalian blood-brain barriers, fewer side effects, etc.) is a drug design approach that is well known in pharmaceutical chemistry.

The term “1-aminocyclohexane derivative” is used herein to describe a compound which is derived from 1-aminocyclohexane (or an available derivative thereof, such as neramexane or memantine) in the process used to create a functionally similar but slightly structurally different drug.

The 1-aminocyclohexane derivatives of the present invention can be represented by the general formula (I):

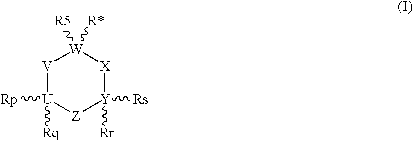

wherein:
R* is —(A)ₙ—(CR¹R²)_{m—NR}³R⁴,
n, m=integers from 0 to 2,
A is selected from the group consisting of linear or branched lower alkyl (C₁-C₆), linear or branched lower alkenyl (C₂-C₆), and linear or branched lower alkynyl (C₂-C₆),
R¹ and R² are independently selected from the group consisting of hydrogen, linear or branched lower alkyl (C₁-C₆), linear or branched lower alkenyl (C₂-C₆), linear or branched lower alkynyl (C₂-C₆) aryl, substituted aryl and arylalkyl,
R³ and R⁴ are independently selected from the group consisting of hydrogen, linear or branched lower alkyl (C₁-C₆), linear or branched lower alkenyl (C₂-C₆), and linear or branched lower alkynyl (C₂-C₆), or together form alkylene (C₂-C₁₀) or alkenylene (C₂-C₁₀) or together with the N form a 3-7-membered azacycloalkane or azacycloalkene, including substituted (alkyl (C₁-C₆), alkenyl (C₂-C₆)) 3-7-membered azacycloalkane or azacycloalkene; or independently R³ or R⁴ may join with R^{p}, R^{q}, R^{r}, or R^{s} to form an alkylene chain —CH(R⁶)—(CH₂)ₜ—,
wherein t=0 or 1 and the left side of the alkylene chain is attached to U or Y and the right side of the alkylene chain is attached to N and R⁶ is selected from the group consisting of hydrogen, linear or branched lower alkyl (C₁-C₆), linear or branched lower alkenyl (C₂-C₆), linear or branched lower alkynyl (C₂-C₆), aryl, substituted aryl and arylalkyl; or independently R³ or R⁴ may join with R⁵ to form an alkylene chain represented by the formula —CH₂—CH₂—CH₂—(CH₂)ₜ—, or an alkenylene chain represented by the formulae —CH═CH—CH₂—(CH₂)ₜ—, —CH═C═CH—(CH₂)ₜ— or —CH₂—CH═CH—(CH₂)ₜ—, wherein t=0 or 1, and the left side of the alkylene or alkenylene chain is attached to W and the right side of the alkylene ring is attached to N;
R⁵ is independently selected from the group consisting of hydrogen, linear or branched lower alkyl (C₁-C₆), linear or branched lower alkenyl (C₂-C₆), and linear or branched lower alkynyl (C₂-C₆), or R⁵ combines with the carbon to which it is attached and the next adjacent ring carbon to form a double bond,
R^{p}, R^{q}, R^{r}, and R^{s}, are independently selected from the group consisting of hydrogen, linear or branched lower alkyl (C₁-C₆), linear or branched lower alkenyl (C₂-C₆), linear or branched lower alkynyl (C₂-C₆), cycloalkyl (C₃-C₆) and aryl, substituted aryl and arylaklyl or R^{p}, R^{q}, R^{r}, and R^{s} independently may form a double bond with U or with Y or to which it is attached, or R^{p}, R^{q}, R^{r}, and R^{s} may combine together to represent a lower alkylene (CH₂)ₓ— or a lower alkenylene bridge wherein x is 2-5, inclusive, which alkylene bridge may, in turn, combine with R⁵ to form an additional lower alkylene —(CH₂)_{y}— or a lower alkenylene bridge, wherein y is 1-3, inclusive,
the symbols U, W, and Y represent carbon atoms, the symbols V, X and Z represent —(CH₂)—, and include optical isomers, diastereomers, enantiomers, solvates, hydrates, pharmaceutically acceptable salts, and mixtures of compounds within formula (I).
The ring defined by U-V-W-X-Y-Z is preferably selected from the group consisting of cyclohexane, cyclohex-2-ene, cyclohex-3-ene, cyclohex-1,4-diene, cyclohex-1,5-diene, cyclohex-2,4-diene, and cyclohex-2,5-diene,
Non-limiting examples of 1-aminocyclohexane derivatives used according to the invention include the 1-aminoalkylcyclohexane derivatives selected from the group consisting of:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane,
N-ethyl-1-amino-1,3,3,5,5-pentamethyl-cyclohexane,
N-(1,3,3,5,5-pentamethylcyclohexyl) pyrrolidine,
3,3,5,5-tetramethylcyclohexylmethylamine,
1-amino-1-propyl-3,3,5,5-tetramethylcyclohexane,
1 amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3(cis)-propylcyclohexane,
1-amino-1,3-dimethyl-3-ethylcyclohexane,
1-amino-1,3,3-trimethylcyclohexane,
cis-3-ethyl-1(trans)-3(trans)-5-trimethylcyclohexamine,
1-amino-1,3(trans)-dimethylcyclohexane,
1,3,3-trimethyl-5,5-dipropylcyclohexylamine,
1-amino-1-methyl-3(trans)-propylcyclohexane,
1-methyl-3(cis)-propylcyclohexylamine,
1-amino-1-methyl-3(trans)-ethylcyclohexane,
1-amino-1,3,3-trimethyl-5(cis)-ethylcyclohexane,
1-amino-1,3,3-trimethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-1-amino-1,3,3,5.5-pentamethylcyclohexane,
1-amino-1-methylcyclohexane,
N,N-dimethyl-1-amino-1,3,3,5,5-pentamethylcyclohexane,
2-(3,3,5,5-tetramethylcyclohexyl)ethylamine,
2-methyl-1-(3,3,5,5-tetramethylcyclohexyl)propyl-2-amine,
2-(1,3,3,5,5-pentamethylcyclohexyl)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,5S)trans-5-ethyl-1,3,3-trimethylcyclohexane,
1-amino-(1S,5S)cis-5-ethyl-1,3,3-trimethylcyclohexane,
1-amino-1,5,5-trimethyl-3(cis)-isopropyl-cyclohexane,
1-amino-1,5,5-trimethyl-3(trans)-isopropyl-cyclohexane,
1-amino-1-methyl-3(cis)-ethyl-cyclohexane,
1-amino-1-methyl-3(cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-1-ethyl-3,3,5,5-tetramethylcyclohexane,
N-ethyl-1-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,5S)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,5S)trans-5-ethyl,3,3-trimethylcyclohexyl]pyrrolidine or piperidine,
N-(1-ethyl-3,3,5,5-tetramethylyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,
   their optical isomers, diastereomers, enantiomers, solvates, hydrates, their pharmaceutically acceptable salts, and mixtures thereof.

Neramexane (1-amino-1,3,3,5,5-pentamethylcyclohexane) is disclosed in U.S. Pat. No. 6,034,134 which is incorporated here by reference.

Certain 1-aminocyclohexane derivatives of general formula (I) including the case where three axial alkyl substituent, e.g., R^{p}, R^{r} and R⁵ all together form a bridgehead to yield compounds (so called 1-aminoadamantanes) illustrated by the formulae IIb-IId below:

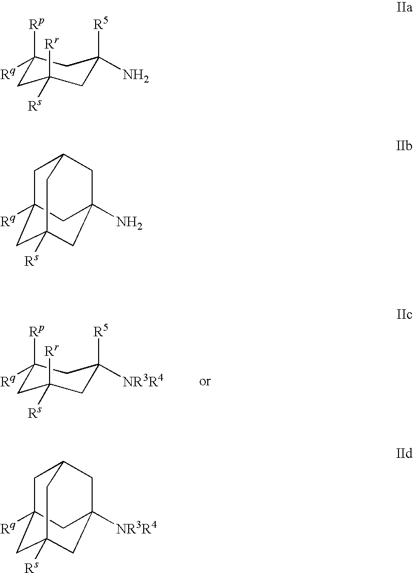

Certain 1-aminocyclohexane derivatives of formula (I) wherein n+m=0, U, V, W, X, Y and Z form a cyclohexane ring, and one or both of R³ and R⁴ are independently joined to said cyclohexane ring via alkylene bridges formed through R^{p}, R^{q}, R^{r}, R^{s} or R⁵ are represented by the following formulae IIIa-IIIc:

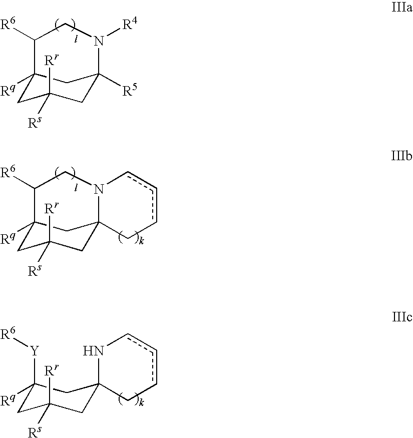

where R^{q}, R^{r}, R^{s}, R^{r} and R⁵ are as defined above for formula (I), R⁶ is hydrogen, linear or branched lower alkyl (C₁-C₆), linear or branched lower alkenyl (C₂-C₆), linear or branched lower alkynyl (C₂-C₆), aryl, substituted aryl or arylalkyl Y is saturated or may combine with R⁶ to form a carbon-hydrogen bond with the ring carbon to which it is attached, I=0 or 1 and k=0, 1 or 2 and [symbol] represents a single or double bond.

Non-limiting examples of 1-aminocyclohexane derivatives used according to the invention include 1-amino adamantane and its derivatives selected from the group consisting of:
1-amino-3-phenyl adamantane,
1-amino-methyl adamantane,
1-amino-3,5-dimethyl adamantane (memantine),
1-amino-3-ethyl adamantane,
1-amino-3-isopropyl adamantane,
1-amino-3-n-butyl adamantane,
1-amino-3,5-diethyl adamantane,
1-amino-3,5-diisopropyl adamantane,
1-amino-3,5-di-n-butyl adamantane,
1-amino-3-methyl-5-ethyl adamantane,
1-N-methylamino-3,5-dimethyl adamantane,
1-N-ethylamino-3,5-dimethyl adamantane,
1-N-isopropyl-amino-3,5-dimethyl adamantane,
1-N,N-dimethyl-amino-3,5-dimethyl adamantane,
1-N-methyl-N-isopropyl-amino-3-methyl-5-ethyl adamantane,
1-amino-3-butyl-5-phenyl adamantane,
1-amino-3-pentyl adamantane,
1-amino-3,5-dipentyl adamantane,
1-amino-3-pentyl-5-hexyl adamantane,
1-amino-3-pentyl-5-cyclohexyl adamantane,
1-amino-3-pentyl-5-phenyl adamantane,
1-amino-3-hexyl adamantane,
1-amino-3,5-dihexyl adamantane,
1-amino-3-hexyl-5-cyclohexyl adamantane,
1-amino-3-hexyl-5-phenyl adamantane,
1-amino-3-cyclohexyl adamantane,
1-amino-3,5-dicyclohexyl adamantane,
1-amino-3-cyclohexyl-5-phenyl adamantane,
1-amino-3,5-diphenyl adamantane,
1-amino-3,5,7-trimethyl adamantane,
1-amino-3,5-dimethyl-7-ethyl adamantane,
1-amino-3,5-diethyl-7-methyl adamantane,
1-N-pyrrolidino and 1-N-piperidine derivatives,
1-amino-3-methyl-5-propyl adamantane,
1-amino-3-methyl-5-butyl adamantane,
1-amino-3-methyl-5-pentyl adamantane,
1-amino-3-methyl-5-hexyl adamantane,
1-amino-3-methyl-5-cyclohexyl adamantane,
1-amino-3-methyl-5-phenyl adamantane,
1-amino-3-ethyl-5-propyl adamantane,
1-amino-3-ethyl-5-butyl adamantane,
1-amino-3-ethyl-5-pentyl adamantane,
1-amino-3-ethyl-5-hexyl adamantane,
1-amino-3-ethyl-5-cyclohexyl adamantane,
1-amino-3-ethyl-5-phenyl adamantane,
1-amino-3-propyl-5-butyl adamantane,
1-amino-3-propyl-5-pentyl adamantane,
1-amino-3-propyl-5-hexyl adamantane,
1-amino-3-propyl-5-cyclohexyl adamantane,
1-amino-3-propyl-5-phenyl adamantane,
1-amino-3-butyl-5-pentyl adamantane,
1-amino-3-butyl-5-hexyl adamantane,
1-amino-3-butyl-5-cyclohexyl adamantane,
   their optical isomers, diastereomers, enantiomers, solvates, hydrates, N-methyl, N,N-dimethyl, N-ethyl, N-propyl derivatives, their pharmaceutically acceptable salts, and mixtures thereof.

Memantine (1-amino-3,5-dimethyl adamantane), for example, is the subject matter of U.S. Pat. Nos.: 4,122,193, 4,273,774, 5,061,703, and 5,614,560.

The 1-amino adamantane derivatives of formulae IIb and IId, including memantine, are generally prepared by alkylation of halogenated adamantanes, preferably bromo- or chloroadamantanes. The di- or tri-substituted adamantanes are obtained by additional halogenation and alkylation procedures. The amino group is introduced either by oxidation with chromiumtrioxide and bromination with HBr or bromination with bromine and reaction with formamide followed by hydrolysis. The amino function can be alkylated according to generally-accepted methods. Methylation can, for example, be effected by reaction with chloromethyl formate and subsequent reduction. The ethyl group can be introduced by reduction of the respective acetamide. For more details on synthesis see, e.g., U.S. Pat. Nos. 5,061,703 and 6,034,134. Additional synthetic techniques for the foregoing compounds can be found in U.S. Published Applications No. 20030166634 and 20040034055, all incorporated by reference.

Various salts and isomers (including stereoisomers and enantiomers) of the drugs listed herein can be used. The term “salts” can include salts of free acids or free bases. Examples of acids which may be employed to form pharmaceutically acceptable acid addition salts include inorganic acids such as hydrochloric, sulfuric, or phosphoric acid, and organic acids such as acetic, maleic, succinic, or citric acid, etc. All of these salts (or other similar salts) may be prepared by conventional means. The nature of the salt or isomer is not critical, provided that it is non-toxic and does not substantially interfere with the desired pharmacological activity.

The term “therapeutically effective” applied to dose or amount refers to that quantity of a compound or pharmaceutical composition that is sufficient to result in a desired activity upon administration to a mammal in need thereof. As used herein with respect to the pharmaceutical compositions comprising an 1-aminocyclohexane derivative, the term “therapeutically effective amount/dose” is used interchangeably with the term “neurologically effective amount/dose” and refers to the amount/dose of a compound or pharmaceutical composition that is sufficient to produce an effective neurological response upon administration to a mammal.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” applied to pharmaceutical compositions of the invention refers to a diluent, excipient, or vehicle with which an active compound (e.g., an 1-aminocyclohexane derivative) is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18^{th} Edition.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

In conjunction with the methods of the present invention, also provided are pharmaceutical compositions comprising a therapeutically effective amount of a 1-aminocyclohexane derivative. The compositions of the invention further can comprise a carrier or excipient (all pharmaceutically acceptable). The compositions can be formulated for once-a-day administration or twice-a-day administration. The preferred 1-aminocyclohexane derivatives are memantine and neramexane.

Memantine (NAMENDA™) is commercially available as the hydrochloride salt in 5 or 10 mg film-coated tablets. However, according to the present invention, the dosage form of memantine may be a solid, semisolid or liquid formulation according to the following.

The 1-aminocyclohexane derivative may be administered orally, topically, parenterally, or mucosally (e.g., buccally, by inhalation, or rectally) in dosage unit formulations containing conventional non-toxic pharmaceutically acceptable carriers. In one embodiment for the administration to pediatric subjects, memantine is formulated as a flavored liquid, e.g., peppermint flavor. The 1-aminocyclohexane derivative may be administered orally in the form of a capsule, a tablet, or the like, or as a semi-solid or liquid formulation (see Remington's Pharmaceutical Sciences, Mack 5 Publishing Co., Easton, Pa.).

For oral administration in the form of a tablet or capsule, the 1-aminocyclohexane derivative can be combined with a non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

The tablets can be coated with a concentrated sugar solution which may contain e.g., gum arabic, gelatine, talcum, titanium dioxide, and the like. Alternatively, the tablets can be coated with a polymer that dissolves in a readily volatile organic solvent or mixture of organic solvents. In specific embodiments, memantine is formulated in to immediate-release (IR) or modified-release (MR) tablets. Immediate release solid dosage forms permit the release of most or all of the active ingredient over a short period of time, such as 60 minutes or less, and make rapid absorption of the drug possible. Modified release solid oral dosage forms permit the sustained release of the active ingredient over an extended period of time in an effort to maintain therapeutically effective plasma levels over similarly extended time intervals and/or to modify other pharmacokinetic properties of the active ingredient.

For the formulation of soft gelatin capsules, the active substances may be admixed with e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatine capsules.

The compositions of the invention can also be introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO 95/11010 and WO 93/07861). Biocompatible polymers may be used in achieving controlled release of a drug, include for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

Memantine-coated non-pareil beads or seeds can also be used according to the present invention (see Huang et al., Drug Dev Ind Pharm. 2002; 28(5):593-9; and Ganesan et al., Boll Chim Farm. 2003;142(7):290-4).

Formulation of the 1-aminocyclohexane derivative in a semi-solid or liquid form wherein the active ingredient, i.e. the 1-aminocyclohexane, is highly soluble in aqueous media may also be used. The active ingredient may constitute between 0.1 and 99% by weight of the formulation, more specifically between 0.5 and 20% by weight for formulations intended for injection and between 0.2 and 50% by weight for formulations suitable for oral administration.

In one embodiment of the invention, the 1-aminocyclohexane derivative is administered in a modified release formulation. Modified release dosage forms provide a means for improving patient compliance and for ensuring effective and safe therapy by reducing the incidence of adverse drug reactions. Compared to immediate release dosage forms, modified release dosage forms can be used to prolong pharmacologic action after administration, and to reduce variability in the plasma concentration of a drug throughout the dosage interval, thereby eliminating or reducing sharp peaks. Modified release dosage forms are described in U.S. patent application Ser. No. 11/155,330, incorporated by reference.

A modified release form dosage can comprise a core either coated with or containing a drug. The core being is then coated with a release modifying polymer within which the drug is dispersed. The release modifying polymer disintegrates gradually, releasing the drug over time. Thus, the outer-most layer of the composition effectively slows down and thereby regulates the diffusion of the drug across the coating layer when the composition is exposed to an aqueous environment, i.e. the gastrointestinal tract. The net rate of diffusion of the drug is mainly dependent on the ability of the gastric fluid to penetrate the coating layer or matrix and on the solubility of the drug itself.

In another embodiment of the invention, the 1-aminocyclohexane derivative is formulated in an oral, liquid formulation. Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound. A particular example of an oral time-controlled release pharmaceutical formulation is described in U.S. Pat. No. 5,366,738.

For oral administration in liquid form, the 1-aminocyclohexane derivative can be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms. For example, solutions may contain from about 0.2% to about 20% by weight of memantine, with the balance being sugar and mixture of ethanol, water, glycerol and propylene glycol. Optionally such liquid formulations may contain coloring agents, flavoring agents, saccharine and carboxymethyl-cellulose as a thickening agent or other excipients.

In another embodiment, a therapeutically effective amount of the 1-aminocyclohexane derivative is administered in an oral solution containing a preservative, a sweetener, a solubilizer, and a solvent. The oral solution may include one or more buffers, flavorings, or additional excipients. In a further embodiment, a peppermint or other flavoring is added to the 1-aminocyclohexane derivative oral liquid formulation.

For administration by inhalation, the 1-aminocyclohexane derivative can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Solutions for parenteral applications by injection can be prepared in an aqueous solution of a water-soluble pharmaceutically acceptable salt of the active substances, preferably in a concentration of from about 0.5% to about 10% by weight. These solutions may also contain stabilizing agents and/or buffering agents and may conveniently be provided in various dosage unit ampoules.

Dosage units for rectal application can be solutions or suspensions or can be prepared in the form of suppositories or retention enemas comprising the 1-aminocyclohexane derivative in a mixture with a neutral fatty base, or gelatin rectal capsules comprising the active substances in admixture with vegetable oil or paraffin oil.

The formulations of the invention can be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. Alternatively, the active ingredient can be in powder form for reconstitution with a suitable vehicle, e.g., sterile pyrogen-free water, before use. For parental administration, the rate of infusion must be carefully controlled due to the relatively long half-life of me the 1-aminocyclohexane derivative mantine in the blood stream.

The invention also provides a pharmaceutical pack or kit comprising one or more containers containing the 1-aminocyclohexane derivative and, optionally, more of the ingredients of the formulation. In a specific embodiment, memantine is provided as an oral solution (2 mg/ml) for administration with the use of a 2 teaspoon capacity syringe (dosage KORC®). Each oral syringe has blue hatch marks for measurement, with lines on the right side of the syringe (tip down) representing tsp units, and those on the left representing ml units.

The optimal therapeutically effective amount should be determined experimentally, taking into consideration the exact mode of administration, from in which the drug is administered, the indication toward which the administration is directed, the subject involved (e.g., body weight, health, age, sex, etc.), and the preference and experience of the physician or veterinarian in charge.

Toxicity and therapeutic efficacy of the compositions of the invention can be determined by standard pharmaceutical procedures in experimental animals, e.g., by determining the LD50 (the dose lethal to 50% of the population) and the ED50 (the dose therapeutically effective in 50% of the population). The dose ratio between therapeutic and toxic effects is the therapeutic index and it can be expressed as the ratio ED50/LD50. Compositions that exhibit large therapeutic indices are preferred.

Suitable daily doses of the active compounds of the invention in therapeutic treatment of humans are about 0.01-10 mg/kg bodyweight on peroral administration and 0.001-10 mg/kg bodyweight on parenteral administration. For adults, suitable daily doses of memantine or neramexane are within the range from about 1.25 mg to about 100 mg per day, preferably, from about 20 to about 40 mg per day. For pediatric subjects aged 4-14, it is preferred that memantine is administered as an oral, liquid dosage form, at about 0.5 mg/day, up to a maximum dose of 10 mg/day. Titration to the maximum dose over about 4 weeks from a lower initial starting dose, e.g., about 1.25 mg/day, with weekly increases by about 1.25 mg/day, is highly recommended. For liquid, oral administration, memantine is dissolved in about one-half the liquid equivalent of the dose. For example, 12.5 mg memantine will be dissolved in 10 ml of the liquid formulation for administration.

Treatment duration can be short-term, e.g., several weeks (for example 8-14 weeks), or long-term until the attending physician deems further administration no longer is necessary.

The 1-aminocyclohexane derivative may be administered as a monotherapy, or in combination with another agent prescribed for the treatment of MS, EL or PBA.

The 1-aminocyclohexane derivative may be administered in combination with an immunomodulator, including glatiramer acetate.

The term “combination” applied to active ingredients is used herein to define a single pharmaceutical composition (formulation) comprising two active agents (e.g., a pharmaceutical composition comprising a 1-aminocyclohexane derivative and an immunomodulator) or two separate pharmaceutical compositions, each comprising an active agent (e.g. a pharmaceutical composition comprising a 1-aminocyclohexane derivative or an immunomodulator), to be administered conjointly.

Within the meaning of the present invention, the term “conjoint administration” is used to refer to administration of the 1-aminocyclohexane and a second active agent (e.g. an immunomodulator) simultaneously in one composition, or simultaneously in different compositions, or sequentially. For the sequential administration to be considered “conjoint”, however, the 1-aminocyclohexane derivative and the second active agent must be administered separated by a time interval which still permits the resultant beneficial effect for treating MS, EL or PBA in a mammal.

### EXAMPLES

The following example illustrates the invention without limiting its scope.

### Example 1

### Double Blind Placebo Controlled Pilot Trial of Memantine for Cognitive Impairment in Multiple Sclerosis

The objective of this pilot project is to conduct a clinical trial to assess the efficacy of memantine as a treatment for cognitive dysfunction in multiple sclerosis (MS). We hypothesize that MS patients with cognitive impairment treated with memantine will demonstrate an improvement in performance on a neuropsychological test battery as compared to placebo treated patients.

Cognitive dysfunction is a major cause of disability in multiple sclerosis. The estimated prevalence of cognitive dysfunction in the MS population is 45% to 65%. MS patients with cognitive dysfunction have fewer social interactions, more sexual dysfunction, greater difficulty with household tasks and higher unemployment than those with normal cognition. At present, there is no effective pharmacological symptomatic treatment for the cognitive dysfunction of MS. One agent that may have some benefit in treating this condition is the N-methyl-D-aspartate (NMDA) receptor antagonist memantine.

Memantine is a NMDA antagonist that has been shown to be effective in treating Alzheimer's disease. Glutamate toxicity has been implicated in the pathogenesis of a variety of neurologic diseases, including MS. Glutamate receptor activation may be involved both in mediation of neural injury and in neuronal dysfunction. By blocking NMDA receptors, memantine may both improve neuronal function, explaining symptomatic improvement in some Alzheimer's patients, and slow progressive neuronal death, potentially resulting in a slowing of cognitive decline in Alzheimer's patients. The pathogenesis of cognitive dysfunction in MS relates as least in part to the extent of cerebral demyelination, axonal loss and atrophy. Some cognitive dysfunction is reversible. Reduction in inflammation can result in improvement in cognitive performance. This investigation will show the role of NMDA receptors and glutamate toxicity may play in the treatment of cognitive dysfunction in MS.

### Study Design

A placebo-controlled, double-blinded, randomized, parallel-group pilot study of 21 weeks duration is planned for MS patients with cognitive impairment. There will be 20 patients per treatment arm. The intervention arm will receive 20 mg of memantine a day. Randomization into each treatment arm will be stratified on age. A double-blind, placebo controlled trial is critical to perform even for a pilot trial. Both learning and placebo effect are likely to improve the cognitive performance of some subjects. An open labeled trial would likely show some improvement in the patients but the results would not be interpretable.

### Statistical Procedures and Populations for Analysis

The Scheduled Visits will be as follows:

Visit 1: The subject will receive consent form. After signing, the visual acuity will be tested. They will receive the first half of the neuropsychological test battery, which includes the Paced Auditory Serial Addition Test (PASAT) and California Verbal Learning Test II (CVLT-II). They will also receive the Beck Depression Inventory (BDI). Women of childbearing potential will be asked to give a urine sample for a pregnancy test (beta HCG). At this point, patients will be informed whether they have met the full criteria for enrollment. If they qualify then they will receive the second half of the neuropsychological tests (Controlled Oral Word Association Test, Stroop Color And Word Association Test, Symbol Digit Modalities Test and Useful Field Of Vision Test).

This visit will last approximately 1½ hours if the patient does not qualify for the study and 2 hours if they qualify.

Visit 2: The subjects will receive the Fatigue Severity Scale (FSS) and Modified Fatigue Impact Scale (MFIS), physical exam and a neurological exam. This visit will last 1 hour.

Visit 3: The complete neuropsychological test battery will be performed. The Medical Outcomes Study 36 Item Short Form Health Survey (SF-36) and the Perceived Deficits Questionnaire (PDQ) from the Multiple Sclerosis Quality of Life Inventory (MSQLI) will be administered. This visit will last 2 hours.

Visit 4: The complete neuropsychological test battery will be administered again. Memantine and placebo pills will be dispensed. The starting dose of memantine will be 5 mg once daily. The dose will be increased in 5 mg increments to 10 mg/day (5 mg twice a day), 15 mg/day (5 mg and 10 mg as separate doses), and 20 mg/day (10 mg twice a day) over 4 weeks and then continued at 20 mg for the rest of the study. This visit will last about 1½ hours.

Telephone follow-up visits will be carried-out for all enrolled subjects four and eight weeks after visit #4. These calls will review study procedures, check for compliance and reports of side effects. The total time for the telephone visit will be 15 minutes.

Visit 5: Subjects will return to clinic for the final assessment 4 weeks after the second telephone follow-up visit. At this visit, subjects will complete the full neuropsychological test battery. The SF-36 and PDQ, BDI, FSS, and MFIS will be administered. A repeat neurological and physical exam will be performed. This visit will last 2½ hours.

Primary Outcome: Neuropsychological Test Battery: Each test will include the neuropsychological test battery which is a commonly used cognitive measure of established validity and reliability. This battery will be tailored to assess those cognitive domains that are most frequently affected in MS. The tests to be included in the battery are the:

1. Paced Auditory Serial Addition Test: A measure of auditory information processing speed.

2. California Verbal Learning Test II: A measure of verbal learning/memory.

3. Controlled Oral Word Association Test: A measure of phonemic fluency.

4. Stroop Color and Word Test: A measure of concentration and attention.

5. Symbol Digit Modalities Test: A measure of information processing speed and visual tracking.

6. Useful Field of Vision Test: A test of visual information processing and divided attention.

Secondary Outcomes:

1. Fatigue Severity Scale: a fatigue severity scale that has used in MS clinical trials.

2. Modified Fatigue Impact Scale: alternate fatigue scale with MS specific items.

3. MS Quality of Life Inventory: An MS-specific health-related quality of life instrument. Only the health survey questionnaire (SF-36) and the Perceived Deficit Questionnaire will be used from the MSQLI.

4. Beck Depression Inventory: a frequently used measure of depression.

Inclusion Criteria:

1. A diagnosis of multiple sclerosis as defined by the McDonald criteria. Patients with relapsing-remitting, secondary progressive, and primary progressive forms of MS will be eligible.

2. Age between 18 and 60 years.

3. Demonstrated cognitive dysfunction at screening defined as a score in the range of 0.5 to 2.5 standard deviations below the mean on the PASAT or the CVLT-II.

Exclusion Criteria:

1. A history of major depression, psychosis, or a score >19 on the Beck's Depression Inventory.

2. Corrected binocular visual acuity worse than 20/50; any impairment of binocular color vision.

3. Patients that do not speak English as a primary language (fluency may impact performance).

4. A clinically significant MS exacerbation within 30 days of the screening

5. Pregnancy

6. Renal insufficiency.

7. History of seizures.

8. Patients using acetazolamide (Diamox, Ak-sol, Storzolamide), dichlorphenamide (Daranide), methazolamide (Neptazane) or topiramate (Topamax), dextrometorphan (Robitussin DM and other cold remedies), amantadine (Symmetrel).

### Data Analysis

The two groups (memantine and placebo) will be compared for randomization inequity for demographic and disease severity factors. Because of possible effects of learning, the third battery will be considered as baseline. The primary analysis will use a repeated measure ANOVA comparing the response in the two groups. A two-sided alpha value of 0.05 is defined as statistical significance. All primary outcome and secondary variables will be analyzed.

### Discussion

It is anticipated that the memantine treated group will show an improvement in performance on the cognitive test battery as compared to the placebo group.

### Example 2

### Use of Neramexane in the EAE Model in Mice

Induction of EAE. Induction of EAE in animal models is well known in the art. (Raine, Chapter 16, Handbook of Clinical Neurology, vol. 3(47): Demyelinating Diseases, Koetsier, ed., (Elsevier Science Publishers 1985). In the present protocol, 6-8 week ABH mice are immunized with mouse spinal cord homogenate in Freund's complete adjuvant on day 0 & 7. (Baker et al. 1990. J. Neuroimmunol 28:261 O'Neill et al 1992. J. Neuroimmunol. 38:53). Animals will develop relapsing remitting episodes of paralysis at approximately 3-4 week intervals. These will be monitored daily from day 11 onwards to ensure severity levels of paralysis and maintained with humane limits according to the Home Office, Animals (Scientific Procedures) Act (1981). Spasticity (stiff hind limbs) after 2-3 relapse episodes (approximately 80-120 days post-induction) will be allowed to develop. Animals with visually spastic limbs will be selected for assessment of the TEST compound.

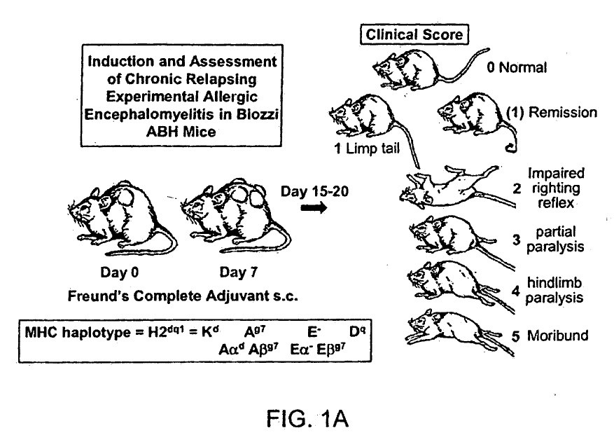

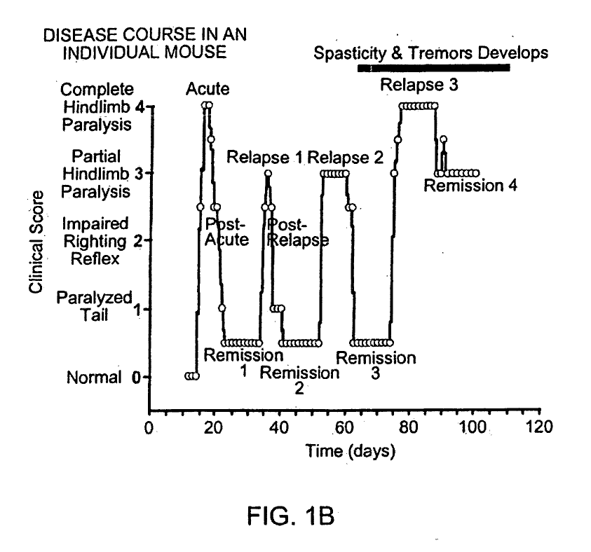

Resistance to flexion against a strain gauge will be the primary assessment (Baker et al. 2000. Nature), secondary outcome measures will be the presence or absence of spastic tails. See FIGS. 1A and 1B.

Spasticity. The “stiffness” of spastic limbs is measured using a purpose built strain gauge, and assessed by the resistance force against hindlimb flexion. The limb is extended manually twice prior to measurement of each limb. Limbs showing severe crossing or flexion will not be analyzed. The signal is amplified and digitized using DAS16 card (Amplicon, Brighton UK.) and acquired using Dacquire V10 software and analyzed using Spike 2 software (Baker et al 2000). Left and right hindlimbs are analyzed alternately and typically 5-8 readings per limb were measured at each time point. The mean values will be calculated and converted to forces in Newtons. Data will be analyzed using one-way repeated measures analysis of variance (ANOVA) incorporating Student-Newman-Keuls posthoc test. Differences to baseline will also be compared using Paired t tests.

Administration of Compound. The TEST compounds are dissolved in vehicle (to be supplied by the SPONSOR-preferably this will be a saline solution) using doses (to be supplied by the SPONSOR). These will be delivered by the intravenous, intraperitoneal or oral route (to be supplied by the sponsor). Resistance to flexion will be assessed at 10 minutes 30 minutes and 60 minutes following intravenous, intraperitoneal injection, 30, 60 and 90 minutes after oral administration. (Escalating doses (up to three) may be used in the same cohort of animals following wash-out to allow for direct comparison of the dose response).

This may be repeated in drug naive animals and the longevity of the effect assessed at hourly intervals up to X hours plus at 24 hours after administration.

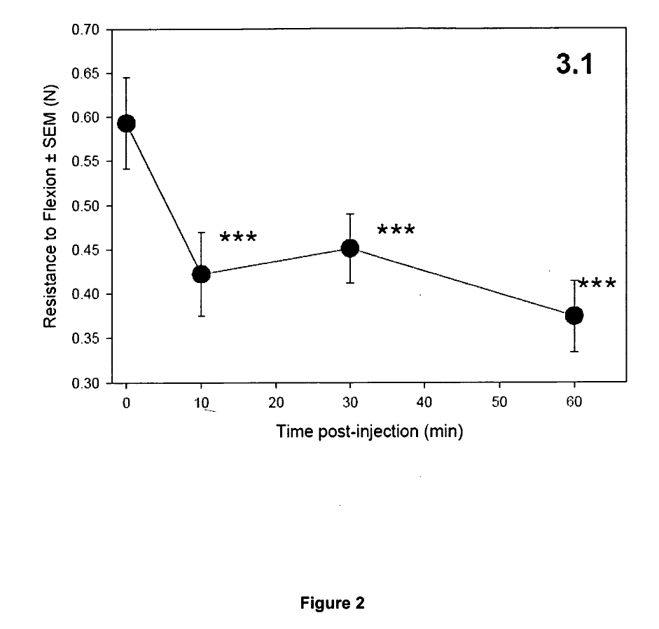

Results—3.1 mg/kg. See FIG. 2.

One Way Repeated Measures Analysis of Variance

Normality Test: Passed (P = 0.043)
Equal Variance Test: Passed (P = 0.448)

| Treatment Name | N | Missing | Mean | Std Dev | SEM |
|---|---|---|---|---|---|
| 0 minN | 13 | 0 | 0.593 | 0.186 | 0.0517 |
| 10 minN | 13 | 0 | 0.422 | 0.171 | 0.0475 |
| 30 minN | 13 | 0 | 0.451 | 0.141 | 0.0392 |
| 60 minN | 13 | 0 | 0.374 | 0.143 | 0.0398 |

| Source of Variation | DF | SS | MS | F | P |
|---|---|---|---|---|---|
| Between Subjects | 12 | 0.906 | 0.0755 | | |
| Between Treatments | 3 | 0.345 | 0.115 | 11.844 | <0.001 |
| Residual | 36 | 0.349 | 0.00970 | | |
| Total | 51 | 1.599 | | | |

The differences in the mean values among the treatment groups are greater than would be expected by chance; there is a statistically significant difference (P=<0.001). To isolate the group or groups that differ from the others use a multiple comparison procedure.

Power of performed test with alpha=0.050:0.999

All Pairwise Multiple Comparison Procedures (Student-Newman-Keuls Method):

Comparisons for factor:

| | Diff of | | | | |
|---|---|---|---|---|---|
| Comparison | Means | p | q | P | P < 0.050 |
| ominN vs. 60 minN | 0.218 | 4 | 7.997 | <0.001 | Yes |
| ominN vs. 10 minN | 0.171 | 3 | 6.262 | <0.001 | Yes |
| ominN vs. 30 minN | 0.142 | 2 | 5.188 | <0.001 | Yes |
| 30 minN vs. 60 minN | 0.0767 | 3 | 2.808 | 0.130 | No |
| 30 minN vs. 10 minN | 0.0293 | 2 | 1.074 | 0.453 | Do Not Test |
| 10 minN vs. 60 minN | 0.0474 | 2 | 1.734 | 0.228 | Do Not Test |

A result of “Do Not Test” occurs for a comparison when no significant difference is found between two means that enclose that comparison. For example, if you had four means sorted in order, and found no difference between means 4 vs. 2, then you would not test 4 vs. 3 and 3 vs. 2, but still test 4 vs. 1 and 3 vs. 1 (4 vs. 3 and 3 vs. 2 are enclosed by 4 vs. 2: 4 3 2 1). Note that not testing the enclosed means is a procedural rule, and a result of Do Not Test should be treated as if there is no significant difference between the means, even though one may appear to exist.

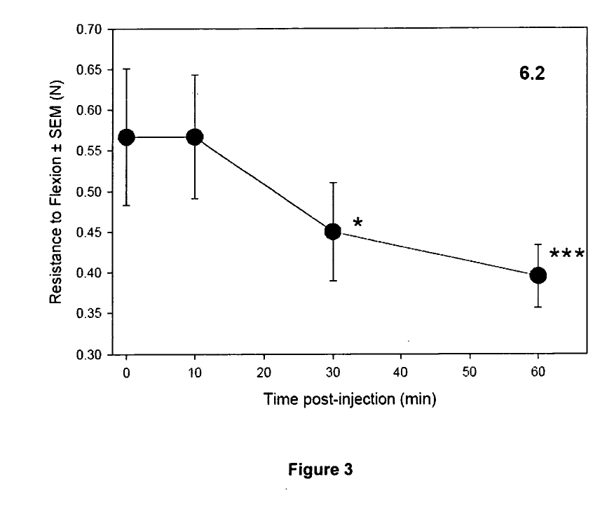

Results—6.2 mg/kg. See FIG. 3.

### One Way Repeated Measures Analysis of Variance

Normality Test: Passed (P > 0.200)
Equal Variance Test: Passed (P = 0.115)

| Treatment Name | N | Missing | Mean | Std Dev | SEM |
|---|---|---|---|---|---|
| 0 min | 11 | 0 | 0.567 | 0.278 | 0.0837 |
| 10 min | 11 | 0 | 0.567 | 0.253 | 0.0763 |
| 30 min | 11 | 0 | 0.450 | 0.201 | 0.0607 |
| 60 min | 11 | 0 | 0.395 | 0.127 | 0.0384 |

| Source of Variation | DF | SS | MS | F | P |
|---|---|---|---|---|---|
| Between Subjects | 10 | 1.735 | 0.173 | | |
| Between Treatments | 3 | 0.246 | 0.0819 | 10.146 | <0.001 |
| Residual | 30 | 0.242 | 0.00807 | | |
| Total | 43. | 2.223 | | | |

The differences in the mean values among the treatment groups are greater than would be expected by chance; there is a statistically significant difference (P=<0.001). To isolate the group or groups that differ from the others use a multiple comparison procedure.

Power of performed test with alpha=0.050: 0.994.

All Pairwise Multiple Comparison Procedures (Student-Newman-Keuls Method):

Comparisons for factor:

| Comparison | Diff of Means | p | q | P | P < 0.050 |
|---|---|---|---|---|---|
| 0 min vs. 60 min | 0.172 | 4 | 6.342 | <0.001 | Yes |
| 0 min vs. 30 min | 0.117 | 3 | 4.316 | 0.013 | Yes |
| 0 min vs. 10 min | 0.0000691 | 2 | 0.00255 | 0.999 | No |
| 10 min vs. 60 min | 0.172 | 3 | 6.340 | <0.001 | Yes |
| 10 min vs. 30 min | 0.117 | 2 | 4.314 | 0.005 | Yes |
| 30 min vs. 60 min | 0.0549 | 2 | 2.026 | 0.162 | No |

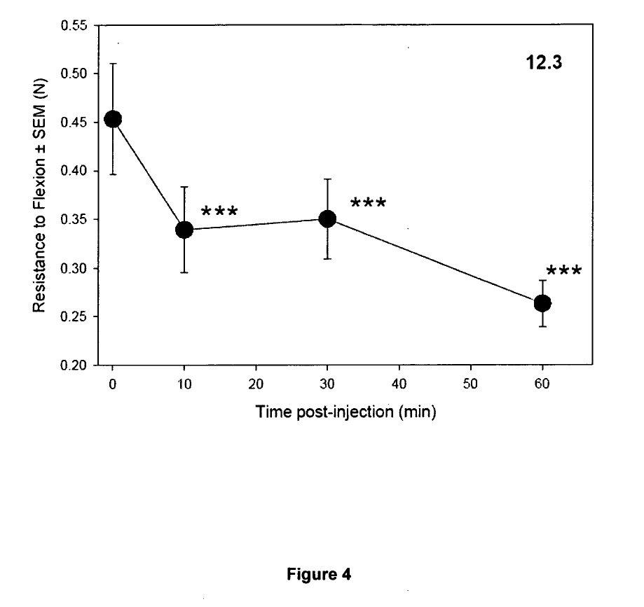

Results —12.3 mg/kg. See FIG. 4.

### One Way Repeated Measures Analysis of Variance

Normality Test: Passed (P = 0.013)
Equal Variance Test: Passed (P = 0.928)

| Treatment Name | N | Missing | Mean | Std Dev | SEM |
|---|---|---|---|---|---|
| 0 min | 14 | 0 | 0.453 | 0.214 | 0.0572 |
| 10 min | 14 | 0 | 0.339 | 0.165 | 0.0441 |
| 30 min | 14 | 0 | 0.350 | 0.154 | 0.0412 |
| 60 min | 14 | 0 | 0.263 | 0.0892 | 0.0238 |

| Source of Variation | DF | SS | MS | F | P |
|---|---|---|---|---|---|
| Between Subjects | 13 | 1.168 | 0.0898 | | |
| Between Treatments | 3 | 0.255 | 0.0849 | 17.160 | <0.001 |
| Residual | 39 | 0.193 | 0.00495 | | |
| Total | 55 | 1.615 | | | |

The differences in the mean values among the treatment groups are greater than would be expected by chance; there is a statistically significant difference (P=<0.001). To isolate the group or groups that differ from the others use a multiple comparison procedure.

Power of performed test with alpha=0.050: 1.000

All Pairwise -Multiple Comparison Procedures (Student-Newman-Keuls Method)

Comparisons for factor:

| Comparison | Diff of Means | p | q | P | P < 0.050 |
|---|---|---|---|---|---|
| 0 min vs. 60 min | 0.189 | 4 | 10.079 | <0.001 | Yes |
| 0 min vs. 10 min | 0.114 | 3 | 6.050 | <0.001 | Yes |
| 0 min vs. 30 min | 0.103 | 2 | 5.487 | <0.001 | Yes |
| 30 min vs. 60 min | 0.0863 | 3 | 4.592 | 0.007 | Yes |
| 30 min vs. 10 min | 0.0106 | 2 | 0.564 | 0.693 | No |
| 10 min vs. 60 min | 0.0757 | 2 | 4.028 | 0.007 | Yes |

All 3 doses of Neramexane used in the study reduced the level of hind limb spasticity present in the affected mice.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

What is claimed:

1. A method of treating multiple sclerosis, emotional lability or pseudobulbar affect in a subject in need thereof, comprising administering an effective amount of a 1-aminocyclohexane derivative and an immunomodulator.

2. The method of claim 1, wherein the 1-aminocyclohexane derivative and the immunomodulator are administered conjointly.

3. The method of claim 2, wherein the 1-aminocyclohexane derivative and the immunomodulator are administered in a single formulation.

4. The method of claim 1, wherein the 1-aminocyclohexane is represented by the general formula (I): 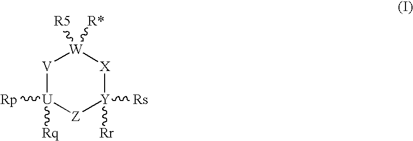
wherein:
R* is —(A)ₙ—(CR¹R²)ₘ—NR³R⁴,
n, m=integers from 0 to 2,
A is selected from the group consisting of linear or branched lower alkyl (C₁-C₆), linear or branched lower alkenyl (C₂-C₆), and linear or branched lower alkynyl (C₂-C₆),
R¹ and R² are independently selected from the group consisting of hydrogen, linear or branched lower alkyl (C₁-C₆), linear or branched lower alkenyl (C₂-C₆), linear or branched lower alkynyl (C₂-C₆) aryl, substituted aryl and arylalkyl, 32 MERZ 54 DIV R³ and R⁴ are independently selected from the group consisting of hydrogen, linear or branched lower alkyl (C₁-C₆), linear or branched lower alkenyl (C₂-C₆), and linear or branched lower alkynyl (C₂-C₆), or together form alkylene (C₂-C₁₀) or alkenylene (C₂-C₁₀) or together with the N form a 3-7-membered azacycloalkane or azacycloalkene, including substituted (alkyl (C₁-C₆), alkenyl (C₂-C₆)) 3-7-membered azacycloalkane or azacycloalkene; or independently R³ or R⁴ may join with R^{p}, R^{q}, R^{r}, or R^{s} to form an alkylene chain —CH(R⁶)—(CH₂)ₜ—,
wherein t=0 or 1 and the left side of the alkylene chain is attached to U or Y and the right side of the alkylene chain is attached to N and R⁶ is selected from the group consisting of hydrogen, linear or branched lower alkyl (C₁-C₆), linear or branched lower alkenyl (C₂-C₆), linear or branched lower alkynyl (C₂-C₆), aryl, substituted aryl and arylalkyl; or independently R³ or R⁴ may join with R⁵ to form an alkylene chain represented by the formula —CH₂—CH₂—CH₂—(CH₂)ₜ—, or an alkenylene chain represented by the formulae —CH═CH—CH₂—(CH₂)ₜ—, —CH═C═CH—(CH₂)ₜ— or —CH₂—CH═CH—(CH₂)ₜ—, wherein t=0 or 1, and the left side of the alkylene or alkenylene chain is attached to W and the right side of the alkylene ring is attached to N;
R⁵ is independently selected from the group consisting of hydrogen, linear or branched lower alkyl (C₁-C₆), linear or branched lower alkenyl (C₂-C₆), and linear or branched lower alkynyl (C₂-C₆), or R⁵ combines with the carbon to which it is attached and the next adjacent ring carbon to form a double bond,
R^{p}, R^{q}, R^{r}, and R^{s}, are independently selected from the group consisting of hydrogen, linear or branched lower alkyl (C₁-C₆), linear or branched lower alkenyl (C₂-C₆), linear or branched lower alkynyl (C₂-C₆), cycloalkyl (C₃-C₆) and aryl, substituted aryl and arylaklyl or R^{p}, R^{q}, R^{r}, and R^{s} independently may form a double bond with U or with Y or to which it is attached, or R^{p}, R^{q}, R^{r}, and R^{s} may combine together to represent a lower alkylene —(CH₂)ₓ— or a lower alkenylene bridge wherein x is 2-5, inclusive, which alkylene bridge may, in turn, combine with R⁵ to form an additional lower alkylene —(CH₂)_{y}— or a lower alkenylene bridge, wherein y is 1-3, inclusive,
the symbols U, W, and Y represent carbon atoms, the symbols V, X and Z represent —(CH₂)—, and include optical isomers, diastereomers, enantiomers, solvates, 33 MERZ 54 DIV hydrates, pharmaceutically acceptable salts, and mixtures of compounds within formula (I).

5. The method of claim 1, wherein the 1-aminocyclohexane derivative is selected from neramexane and prodrugs, salts, isomers, analogs derivatives thereof.

6. The method of claim 1, wherein the immunomodulator is selected from glatiramer acetate and prodrugs, salts, isomers, analogs derivatives thereof.

7. A pharmaceutical composition for treatment multiple sclerosis, emotional lability or pseudobulbar affect, comprising a 1-aminocyclohexane derivative, an immunomodulator and a pharmaceutically acceptable carrier or excipient, wherein the 1-aminocyclohexane derivative and the immunomodulator are present at therapeutically effective dosages.

8. The pharmaceutical composition of claim 7, wherein the 1-aminocyclohexane is represented by the general formula (I): 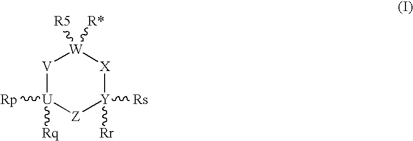
wherein:
R* is —(A)ₙ— (CR¹R²)ₘ—NR³R⁴,
n, m=integers from 0 to 2,
A is selected from the group consisting of linear or branched lower alkyl (C₁-C₆), linear or branched lower alkenyl (C₂-C₆), and linear or branched lower alkynyl (C₂-C₆),
R¹ and R² are independently selected from the group consisting of hydrogen, linear or branched lower alkyl (C₁-C₆), linear or branched lower alkenyl (C₂-C₆), linear or branched lower alkynyl (C₂-C₆) aryl, substituted aryl and arylalkyl,
R³ and R⁴ are independently selected from the group consisting of hydrogen, linear or branched lower alkyl (C₁-C₆), linear or branched lower alkenyl (C₂-C₆), and linear or branched lower alkynyl (C₂-C₆), or together form alkylene (C₂-C₁₀) or alkenylene (C₂-C₁₀) or together with the N form a 3-7-membered azacycloalkane or azacycloalkene, including substituted (alkyl (C₁-C₆), alkenyl (C₂-C₆)) 3-7-membered azacycloalkane or azacycloalkene; or independently R³ or R⁴ may join with R^{p}, R^{q}, R^{r}, or R^{s} to form an alkylene chain —CH(R⁶)—(CH₂)ₜ—,
wherein t=0 or 1 and the left side of the alkylene chain is attached to U or Y and the right side of the alkylene chain is attached to N and R⁶ is selected from the group consisting of hydrogen, linear or branched lower alkyl (C₁-C₆), linear or branched lower alkenyl (C₂-C₆), linear or branched lower alkynyl (C₂-C₆), aryl, substituted aryl and arylalkyl; or independently R³ or R⁴ may join with R⁵ to form an alkylene chain represented by the formula —CH₂—CH₂—CH₂—(CH₂)ₜ—, or an alkenylene chain represented by the formulae —CH═CH—CH₂—(CH₂)ₜ—, —CH═C═CH—(CH₂)ₜ— or —CH₂—CH═CH—(CH₂)ₜ—, wherein t=0 or 1, and the left side of the alkylene or alkenylene chain is attached to W and the right side of the alkylene ring is attached to N;
R⁵ is independently selected from the group consisting of hydrogen, linear or branched lower alkyl (C₁-C₆), linear or branched lower alkenyl (C₂-C₆), and linear or branched lower alkynyl (C₂-C₆), or R⁵ combines with the carbon to which it is attached and the next adjacent ring carbon to form a double bond,
R^{p}, R^{q}, R^{r}, and R^{s}, are independently selected from the group consisting of hydrogen, linear or branched lower alkyl (C₁-C₆), linear or branched lower alkenyl (C₂-C₆), linear or branched lower alkynyl (C₂-C₆), cycloalkyl (C₃-C₆) and aryl, substituted aryl and arylaklyl or R^{p}, R^{q}, R^{r}, and R^{s} independently may form a double bond with U or with Y or to which it is attached, or R^{p}, R^{q}, R^{r}, and R^{s} may combine together to represent a lower alkylene —(CH₂)ₓ— or a lower alkenylene bridge wherein x is 2-5, inclusive, which alkylene bridge may, in turn, combine with R⁵ to form an 35 MERZ 54 DIV additional lower alkylene —(CH₂)_{y}— or a lower alkenylene bridge, wherein y is 1-3, inclusive,
the symbols U, W, and Y represent carbon atoms, the symbols V, X and Z represent —(CH₂)—, and include optical isomers, diastereomers, enantiomers, solvates, hydrates, pharmaceutically acceptable salts, and mixtures of compounds within formula (I).

9. The pharmaceutical composition of claim 7, wherein the 1-aminocyclohexane derivative is selected from neramexane and prodrugs, salts, isomers, analogs derivatives thereof.

10. The pharmaceutical composition of claim 7, wherein the immunomodulator is selected from glatiramer acetate and prodrugs, salts, isomers, analogs derivatives thereof.
